(19) United States
(12) Plant Patent Application Publication
(10) Pub. No.: US 2006/0150290 P1
(43) Pub. Date: Jul. 6, 2006

(54) SCAEVOLA PLANT NAMED 'SCABLHATIS'

(50) SCAEVOLA AEMULA; SCAEVOLA

(75) Inventor: Gabriel Danziger, Moshav Nir Zvi (IL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/030,024

(22) Filed: Jan. 6, 2005

(51) Int. Cl. A01H 5/00

(52) U.S. Cl. PLT/363

(57) ABSTRACT

A new and distinct Scaevola plant named ‘Scablhatis’, characterized by its spreading and cascading growth habit and blue flowers that have a big light yellow eye.

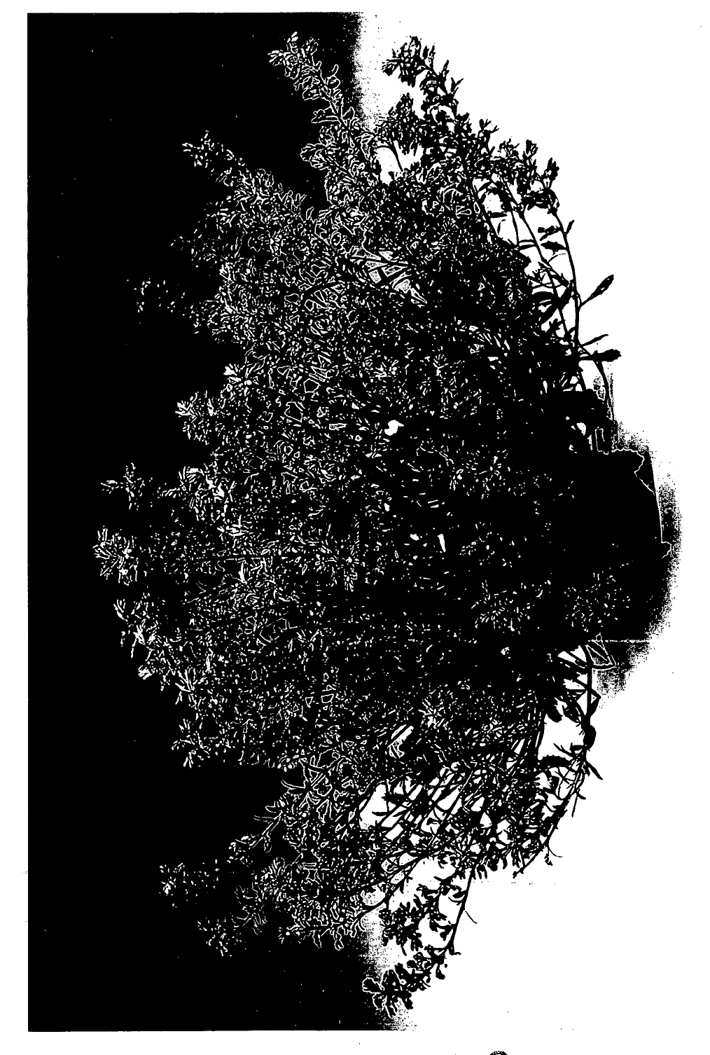

### LATIN NAME OF THE GENUS AND SPECIES OF THE PLANT CLAIMED

Scaevola aemula

### VARIETAL DENOMINATION

‘Scablhatis’

### BACKGROUND OF THE NEW PLANT

The present invention comprises a new and distinct cultivar of Scaevola plant, botanically known as Scaevola aemula, synonym Scaevola saligna and hereinafter referred to by the cultivar name ‘Scablhatis.’

The new cultivar originated from a cross made in 1999 of two selections of Scaevola aemula: ‘SC100’ as female parent and an open pollinated unidentified variety as pollen male parent. Both of these selections are not commercially available and not known by any synonyms.

The new Scaevola plant was selected as a flowering plant within the progeny of this cross in a controlled environment in Enkhuizen, The Netherlands. Plants of the new Scaevola have blue flowers whereas plants of the unidentified selections have purple/blue flowers which is typical for the species Scaevola aemula.

The new Scaevola plant has been asexually reproduced by cuttings in Enkhuizen, The Netherlands and Sarrians, France over a two year period. It has been found to retain its distinctive characteristics through successive propagations, and this novelty appears to be firmly fixed.

### SUMMARY OF THE INVENTION

The new cultivar has not been observed under all possible environmental conditions. The phenotype may vary somewhat with variations in environment such as temperature, day length and light intensity, without, however, any variance in genotype. This description is made under the following greenhouse conditions: day length: from 13 to 15 hours per day, temperature: day 22 degrees Celsius, night 16 degrees Celsius.

The following characteristics have been repeatedly observed and are determined to be basic characteristics of ‘Scablhatis,’ which distinguish ‘Scablhatis’ as a new and distinct cultivar.

1) Low-growing, spreading and cascading growth habit.

2) Medium internodes, dense and bushy plant form and freely branching plant habit.

3) Very freely flowering, more than 600 flowers and flower buds per plant.

4) Unique very large blue fan-shaped flowers with light green-yellow eye.

### DESCRIPTION OF THE DRAWING

This new Scaevola plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Scaevola plant. The data that define these characteristics were collected from asexual reproductions carried out in Enkhuizen, Netherlands. The plant history was taken on 16 week old plants, blossomed under natural light in a greenhouse and grown in a 13 cm container. Colour readings were taken in the greenhouse under ambient light. Colour references are primarily to the RHS Colour Chart of The Royal Horticultural Society of London.

TABLE 1
Differences between the new cultivar ‘Scablhatis’,
its parents and a similar cultivar

| | ‘Scablhatis’ | | ‘SC100’ | | ‘MALE’ | ‘Newon’ | |
|---|---|---|---|---|---|---|---|
| Flower color | Blue | | Blue | | Open | Blue | |
| | with eye | | with eye | | pollination | with eye | |
| Flower size | | | | | | | |
| Length | 2.6-3.2 | cm | 2.5-3 | cm | Open | 3.0-3.6 | cm |
| | | | | | pollination | | |
| Width | 2.8-3.4 | cm | 2.8-3.2 | cm | Open | 3.2-3.8 | cm |
| | | | | | pollination | | |
| Size of | 0.2 | cm | | | Open | 0.4 | cm |
| eye zone | | | | | pollination | | |
| Shoot length | 40-60 | cm | | | Open | 60-90 | cm |
| | | | | | pollination | | |

| | | | |
|---|---|---|---|
| Seed set | Abundant | Open | None |
| | | pollination | |

The most resembling variety ‘Newon’ is patented as U.S. Plant Pat. No. 10,584.
Botanical classification : Scaevola aemula.
Parentage — Female parent: A seedling named ‘SC100’ is one of our seedlings from our A-generation of plants bred in 1998. — Male parent: A seedling named ‘MALE’ is one of our seedlings from our A-generation of plants bred in 1998.

### Propagation

Type cutting: Terminal cuttings.
Time to initiate roots (Summer): About 10-16 days at 24 degrees C.
Time to develop roots (Summer): About 20 days at 24 degrees C.
Root description: Numerous, fine, freely branching.

### The Plant

Plant form and growth habit: Semi-erect, later spreading.
Plant height: About 28 cm.
Plant width (spread): About 68 cm.
Branching character: Freely branching and lateral branching at every node.
Quantity of branches: 40-50 per plant.
Blooming period: From May until November.

### The Stem

Diameter: 2-4 mm.
Shape: Round.
Color: 141B.
Anthocyanin pigmentation: Present, color 176A.
Length of internode: 3.8-5.2 cm.
Texture: Smooth, no pubescence.

### The Foliage

Phyllotaxis: Alternate, single.
Shape of blade: Lanceolate to oblanceolate.
Texture — Upper side: Smooth — Lower side: Smooth.
Anthocyanin pigmentation: Present, color 179C.
Attachment to leaf: Petiolate.
Venation: Pinnate.
Leaf margin: Dentate.
Leaf base: Attenuate.
Leaf apex: Acute.
Length: Gradual size reduction towards lateral branch apex until leaves subtending flowers apprear bract-like.
Length — Lower leaves: 4.1-4.8 cm — Higher leaves: 1.1-1.4 cm.
Width — Lower leaves: 1.9-2.8 cm — Higher leaves: 0.3-0.4 cm.
Depth of incision: 0.2-0.3 cm.
Color — Upper side: 137A — Lower side: 137C.
Petiole — Length: 0.5 cm — Width: 0.3 cm — Color: 137A.

### The Bud

Size of the bud — Length 1.8-2.2 cm — Diameter: 0.2 cm.
Shape: Elliptic — Apex: Acute — Base: Cuneate.
Anthocyanin pigmentation: Present, color 179C.
Color: N144C.
Sepals — Color — Base: N144C — Apex: 143A — Form: Opposite — Number: 2, parted — Size — Length: 0.7 cm — Width: 0.15 cm — Shape: Ligulate — Margin: Entire — Texture: Smooth.

### The Flower

Form: Zygomorphic, semi-circular, fan-shaped with five petals fused at the base to form a tubular flower throat. Flower throat open along the upper surface exposing reproductive organs.
Flower width: 2.8-3.4 cm.
Flower length: 2.6-3.2 cm.
Flower aspect: Horizontal to stem.
Flower arrangement: Solitary, one flower per leaf axil, flowers sessile.
Color — Upper side: Between 92A and 92B — Lower side: 92C — Middle stripe upper side: 92A — Middle stripe lower side: 155D.
Color of the eye: 150C.
Size of the eye: 0.2 cm.
Perianth — Aspect: Fan-shaped, flat — Length, fan: About 2.9 cm — Width, fan: About 3.2 cm — Height: About 1.8 cm.
Inner tube color: 6D.
Outer tube color: 92C.
Overlapping of the petals: Almost separate, fused at the base.
Number of petals: Five.
Shape of the petals: Oblanceolate.
Apex: Mucronate.
Margin: Entire.
Surface texture: Smooth.
Size — Width: 0.5 cm — Length: 1.5 cm.
Number of flowers per lateral branch: 80-100.
Number of flowers at one time open: 6-12.
Fragrance: No fragrance.
Lastingness of the individual flower: 3-4 days.
Lastingness of the bloom: New flowers continue to open in one inflorescence for 30 days.

### The Reproductive Organs

Androecium — Stamen number: Five — Anther shape: Oblong — Anther length: 1-2 mm — Anther diameter: Less than 1 mm — Anther color: 165B — Amount of pollen: Normal — Pollen color: 165B.
Gynoecium — Pistil number: 1 — Pistil length: 1-1.6 cm — Style length: 1 cm — Style color: 144C — Stigma color: 144C — Stigma shape: Oblong — Ovary color: 144C.

### The Seed

From one flower on seed is formed; with one embryo within. Seed set is abundant in later season. From every flower, one seed is produced. The seed is hard coated, so little germination occurs without scarification.
Size — Length: 1.5-2 mm — Width: 1 mm.
Texture of the surface: Smooth.
Color when fallen from plant: 146D.
Color when ripe: 166B.

### Disease/Pest Resistance

No resistance to pests and diseases common to Scaevola (mainly Verticillium and Phtophtora) is observed.

### Temperature tolerance w

Plants of the new Scaevola have been observed to tolerate temperatures from 2 to 30 degrees C.

What is claimed is:

1. A new and distinct Scaevola plant named ‘Scablhatis’, substantially as illustrated and described herein.
